(19) United States
(12) Patent Application Publication
(10) Pub. No.: US 2009/0280114 A1
(43) Pub. Date: Nov. 12, 2009

(54) PREVENTION OF BRAIN INFLAMMATION AS A RESULT OF INDUCED AUTOIMMUNE RESPONSE

(75) Inventor: Beka SOLOMON, Herzilia Pituach (IL)

Correspondence Address: BROWDY AND NEIMARK, P.L.L.C.;624 NINTH STREET, NW, SUITE 300, WASHINGTON, DC, 20001-5303, US

(73) Assignee: Ramot At Tel Aviv University Ltd., Tel Aviv (IL)

(21) Appl. No.: 12/467,931

(22) Filed: May 18, 2009

Related U.S. Application Data
(63) Continuation-in-part of application No. 10/510,820, filed on Aug. 22, 2005, now abandoned.
(60) Provisional application No. 60/371,719, filed on Apr. 12, 2002.

(51) Int. Cl. A61K 39/395; A61P 25/28

(52) U.S. Cl. 424/130.1

(57) ABSTRACT

A disease characterized by amyloid aggregation in a patient may be prevented or treated by causing antibodies against a peptide component of the amyloid deposit to come into contact with the aggregated or soluble amyloid. In order to decrease the risk of inflammation in such a method, the Fc receptors of the patient are blocked, preferably by administration of an effective amount of IVIg, prior to the procedure of causing the antibodies to come into contact with the amyloid.

The application is a continuation-in-part of application Ser. No. 10/510,820, filed Aug. 22, 2005, which was pending upon filing of this application but is now abandoned, which is a 371 National Stage application of international application PCT/US03/11316, filed Apr. 14, 2003, which claims the benefit of 35 USC § 119(e) of provisional application No. 60/371,719, filed Apr. 12, 2002. The entire contents of the above-identified applications are hereby incorporated herein by reference.

### FIELD OF THE INVENTION

The present invention is directed to methods for reducing risk of inflammation as a result of induced autoimmune response and particularly as a result of immunotherapy of diseases characterized by amyloid aggregation.

### BACKGROUND OF THE INVENTION

Methods for the prevention or treatment of diseases characterized by amyloid aggregation in a patient have been proposed which involve causing antibodies against a peptide component of an amyloid deposit to come into contact with aggregated or soluble amyloid. See WO99/27944 of Schenk and U.S. Pat. No. 5,688,651 of Solomon, the entire contents of each of which being hereby incorporated herein by reference. The antibodies may be caused to come into contact with the soluble or aggregated amyloid by either active or passive vaccination. In active vaccination, a peptide, which may be an entire amyloid peptide or a portion thereof, is administered in order to raise antibodies in vivo, which antibodies will bind to the soluble and/or the aggregated amyloid. Passive vaccination involves administering antibodies specific to the amyloid peptide directly. These procedures are preferably used for the treatment of Alzheimer's disease by diminishing the amyloid plaque or slowing the rate of deposition of such plaque.

It has been reported that clinical trials had been undertaken by Elan Corporation and Wyeth-Ayerst Laboratories of a vaccine to test such a process. The compound being tested was AN-1792. This product has been reported to be a form of β-amyloid 42. However, in February of 2002, the two companies announced that the vaccine study had been halted after more than a dozen participants developed severe brain inflammation. In view of the promising prospects of such an immunotherapy program, particularly in light of the animal data set forth in WO99/27944 and Schenk et al (1999), it would be of great benefit to find a way to allow the clinical trials of this immunotherapeutic method to continue without the risk of brain inflammation.

Antibody-antigen complexes initiate the inflammatory response and are central to the pathogenesis of tissue injury. The immune complex triggers inflammation, which is initialized by cell bound Fc receptors, and is then amplified by cellular mediators and activated complement. The accepted model of inflammation is one in which antibodies bind their antigen, forming immune complex, which in turn binds and activates the complement by means of the “classical pathway” (Clynes et al, 1995).

The classical model for this immunopathological cascade, the Arthus reaction, was reinvestigated with a murine strain deficient in Fc receptor expression (Sylvestre et al, 1994). Despite normal inflammatory responses to other stimuli, the inflammatory response to immune complexes was markedly attenuated. These results suggest that the immune complex-triggered inflammation is initiated by cell bound Fc receptors and is then amplified by cellular mediators and activated complement. These results redefine the inflammatory cascade and may offer other approaches for the study and treatment of immunological injury.

Cell membrane receptors specific for the Fc portion of immunoglobulin (FcR) play an important role in immunity and resistance to infection, providing a system that couples antibody-antigen interaction with cellular effector mechanisms. Distinct cell membrane FcRs have been described for all classes of immunoglobulins. The FcRs comprise a multi-membered family of structurally homologous but distinct receptors and are expressed on the vast majority of leukocytes. The diversity of these receptors is reflected in a wide variety of biological responses immediately upon their binding of IgG-antigen complexes, including phagocytosis, endocytosis, antibody-dependent cell-mediated cytotoxicity (ADCC), release of inflammatory mediators and regeneration of B-cell function (Clynes et al, 1995).

### SUMMARY OF THE INVENTION

The present invention solves the problem of increased risk of brain inflammation as a result of induced autoimmune response by eliminating the inflammation pathway initiated by binding of an immune complex to an Fc receptor. The present invention is based on the realization that the brain inflammation that caused the cessation of the clinical trials for AN-1792 was most likely caused by the inflammatory reaction initiated by binding of the immune complex to Fc receptors. This immune reaction could be stopped before it begins by one of two techniques in accordance with the present invention. The first such technique is to block the Fc receptors prior to commencing the immunotherapy. The preferred way to do this is to administer a large dose of IVIg, i.e., human intact intravenously administered immunoglobulin.

Intravenous immunoglobulins (IVIg) have become an established component of immunomodulatory therapy in neurological autoimmune diseases, including inflammatory diseases of the central nervous system (CNS) (van der Meché and van Doorn, 1997; Dalakis, 1999; Stangel et al, 1999). This embodiment of the present invention is based on the realization that IVIg can be used as a preventive step prior to immunotherapy designed to cause antibodies against amyloid-β to come into contact with aggregated or soluble amyloid-β in vivo, regardless of whether the antibodies are directly administered or generated in vivo by administering an antigenic peptide, such as an amyloid peptide.

Since the date of the filing of the original parent application on this invention, it has been shown that administering IVIg alone has the effect of treating Alzheimer's disease (Solomon, 2007; Dodel et al., 2004; Weksler et al., 2005; Relkin et al., 2008; Istrin et al., 2006).

For instance, IVIg was shown in two independent preliminary clinical trials to have several positive effects on patients with Alzheimer's disease. First, it lowered the level of soluble amyloid-β in the CNS. Second, it stabilized cognitive decline, and in several patients it even improved cognitive function (Dodel et al., 2004; Weksler et al., 2004).

The clinical study in Dodel et al., 2004, involved treating five patients having Alzheimer's disease with 1.3 g/kg of IVIg monthly for 6 months (total dose of 0.4 g/kg body weight on three consecutive days every 4 weeks over 6 months). Amyloid-β levels were measured in the CSF and blood, and the effect of treatment on cognition was tested. Amyloid-β levels in CSF decreased (by 30%) over a 6-month period, while total amyloid-β in the serum increased (by 23%) (see the “Results” and “Discussion” on pages 1473-1474 of Dodel et al., 2004). ADAS-cog scores showed cognitive improvement following the treatment, and in several patients it even improved cognition. The results also showed stabilization of cognitive decline as no patient deteriorated (see Table 3 and the “Results” on page 1474 of Dodel et al., 2004; and pages 253-254 and FIG. 5 in Weksler et al., 2005).

Another recent study investigated the mechanism by which IVIg mediates clearing of soluble amyloid-β peptide from the brain of Alzheimer's patients (Istrin et al., 2006). It was found that IVIg dissolves preformed amyloid-β fibrils in vitro and increases cellular tolerance to amyloid-β and mediates phagocytosis of amyloid-β (Istrin et al., 2006).

These findings were confirmed in a recent 18-month clinical study of IVIg therapy (Relkin et al., 2008). In this recent study, it was found that anti-amyloid-β antibodies in the serum from Alzheimer's patients increased in proportion to IVIg dose, and that plasma amyloid-β levels increased transiently after IVIg infusion. Amyloid-β in CSF decreased significantly after six months of IVIg therapy, returned to baseline after a washout period of no therapy and decreased again after IVIg was re-administered. It was also found that cognitive function improved after six months of IVIg therapy, returned to baseline during a washout period of no therapy, and remained stable during subsequent IVIg treatment.

The above recent studies show the effectiveness of IVIg therapy alone for treating Alzheimer's disease. Accordingly, when utilizing the method of the first embodiment of the present invention, one will inherently achieve the improved effect of the above-discussed recent studies, in addition to the effect of reducing the brain inflammation (that caused the cessation of the clinical trials for AN-1792) in the concomitant IVIg therapy with active or passive immunotherapy with anti-amyloid-β antibodies. Thus, not only will one obtain the expected results of treatment of Alzheimer's disease by means of active or passive immunotherapy with anti-amyloid-β antibodies, but one will also achieve results which were unexpected as of the effective filing date of the present application, i.e., reduced inflammation and the direct anti-Alzheimer's effect of IVIg. Regardless of whether or not it would have been obvious that concomitant administration of IVIg with active or passive immunotherapy with amyloid-β antibodies would eliminate inflammation caused by the immunotherapy, one could not have predicted as of the effective filing date of the present application that the concomitant IVIg treatment would have the additional effect of directly treating Alzheimer's disease. This additive effect against Alzheimer's disease would have been totally unexpected for anyone of ordinary skill in the art as of the effective filing date of the present application.

The second method to avoid binding of the immune complex to Fc receptors is to use antibodies that are devoid of Fc regions. Thus, rather than generating intact antibodies in vivo by active vaccination, one would administer antibodies by passive vaccination but using antibodies devoid of Fc regions. Examples of antibodies devoid of Fc regions include Fab, F(ab)₂ and/or scFv antibodies. Such antibodies will still bind to the amyloid or amyloid plaque, but the immune complexes will not start the inflammation sequence because they will not bind to Fc receptors.

While it is believed that the immune complexes using antibodies without an Fc receptor will be cleared by other mechanisms than the Fc receptor mechanism, other means may be provided to effect or promote such clearance. For example, it is known that filamentous phages as a delivery system of scFv and Fab are able to remove the plaque by efflux from brain-blood or other peripheral membranes. Other carrier material for the antibodies devoid of Fc can also be used to promote efflux of the immune complexes.

### DETAILED DESCRIPTIONS OF PREFERRED EMBODIMENTS

### Blocking of Fc Receptors Prior to Immunization by Intravenous Immunoglobulin (IVIg) Administration

Microglial activation is frequently observed in the pathogenesis of neurodegenerative diseases, such as Alzheimer's disease, Parkinson's disease, multiple sclerosis, AIDS dementia complex and amyotrophic lateral sclerosis. In addition, glia, especially microglia, become activated (a process termed reactive gliosis) following an initial wave of neuronal death resulting from traumatic injury, exposure to neurotoxins, and ischemia in the brain. Activated microglia produce a variety of proinflammatory and cytotoxic factors including cytokines. Microglia are very sensitive to changes in the CNS microenvironment and rapidly become activated in virtually all conditions that disrupt normal neuronal functions. Upon activation, microglia secrete a range of immune regulatory peptides as cytokines and non-specific inflammatory mediators, e.g., nitric oxide, and become phagocytic, thus representing the latent scavenger cells of the CNS (Liu et al, 2001).

Controlled trials in multiple sclerosis (MS) and case reports in acute demyelinating encephalomyelitis (ADEM) have shown that intravenous immunoglobulins (IVIg) are of therapeutic benefit in CNS inflammatory diseases. It has been shown that Fc receptor-mediated phagocytosis was inhibited by IVIg, presumably by blockage of the Fc receptor (Stangel et al, 2001). These different effects may protect oligodendrocytes from antibody-mediated phagocytosis and on the other hand could terminate the immune reaction by induction of apoptosis. In accordance with the present invention, IVIg, in addition to known effects on the peripheral immune system, may also be used to modulate the local immune reaction in CNS inflammatory disease.

Similarly, γ-chain-deficient mice are completely resistant to the development of experimental immune thrombocytopenia induced by mouse anti-platelet antibodies. These data support the concept of the present invention that Fc receptors play in integral role in the pathogenesis of type II hypersensitivity and suggest the concomitant potential therapeutic benefits of Fc receptor blockade.

Passive Vaccination Approach May be Based on Delivery (i.p or i.n) of Antibodies Devoid of Fc Regions, Namely Fab, F(ab)₂ and/or scFv.

The laboratory of the present inventor has previously proved that the Fc region is not involved in disaggregation of amyloid plaque, as scFv devoid of Fc performed similarly to whole antibodies in disaggregation of β-amyloid. Accordingly, such antibodies are preferred to intact antibodies as they will not cause initiation of the inflammatory cascade as they will not be bound by Fc receptors.

Phages as a delivery system of scFv and Fab are able to remove the plaque via efflux from brain-blood or other peripheral membranes. Thus, use of a phage delivery system, or any other carrier for the antibody which potentiates efflux of the immune system of the immune complex is a preferred embodiment of the present invention.

If whole antibodies bound to phage are used for i.n. administration, the IVIg injection method for blocking the majority of Fc receptors in microglia, prior to the i.p or i.n. injection of whole antibodies should be undertaken in order to avoid over-activation of microglia.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Clynes et al, “Cytotoxic antibodies trigger inflammation through Fc receptors”, Immunity, 3:21-26 (1995)
Dalakas M C, “Intravenous immunoglobulin in the treatment of autoimmune neuromuscular diseases: present status and practical therapeutic guidelines”, Muscle Nerve 22:1479-1497 (1999)
Dodel et al., “Intravenous immunoglobulins containing antibodies against β-amyloid for the treatment of Alzheimer's disease”, J. Neurol. Neurosurg. Psychiatry, 75:1472-1474 (2004)
Istrin et al., “Intravenous immunoglobulin enhances the clearance of fibrillar amyloid-β peptide”, Journal of Neuroscience Research 84:434-443 (2006)
Liu et al, “Molecular consequences of activated microglia in the brain: overactivation induces apoptosis”, J Neurochem 77:182-189 (2001)
Relkin et al., “18-Month Study of intravenous immunoglobulin for treatment of mild Alzheimer's disease”, Neurobiology of Aging (2008)
Schenk et al, “Immunization with amyloid-beta attenuates Alzheimer-disease-like pathology in the PDAPP mouse”, Nature, 400(6740):116-117 (1999)
Solomon, “Intravenous immunoglobulin and Alzheimer's disease immunotherapy”, Current Opinion in Molecular Therapeutics, 9(1):79-85 (2007)
Stangel et al, “Mechanisms of high-dose intravenous immunoglobulins in demyelinating diseases”, Arch Neurol 56:661-663 (1999)
Stangel et al, “Polyclonal immunoglobulins (IVIg) modulate nitric oxide production and microglial functions in vitro via Fc receptors”, J Neuroimmunol 112:63-71 (2001)
Sylvestre et al, “Fc receptors initiate the Arthus reaction: redefining the inflammatory cascade”, Science, 265:1095 (1994)
van der Meché and van Doorn, “The current place of high-dose immunoglobulins in the treatment of neuromuscular disorders”, Muscle Nerve 20:136-147 (1997)
Weksler et al., “The immune system, amyloid-β peptide, and Alzheimer's disease, Immunological Reviews, 205:244-256 (2005)

1. A method for preventing inflammation caused by immunotherapy treatment in which antibodies against a peptide component of an amyloid deposit are caused to come into contact with aggregated or soluble amyloid, comprising, prior to any such immunotherapy treatment:
intravenously administering to a patient having a disease characterized by amyloid aggregation, an effective amount of immunoglobulin (IVIg).

2. A method in accordance with claim 1, wherein the IVIg is intact human polyclonal immunoglobulin.

3. A method in accordance with claim 1, wherein the disease characterized by amyloid aggregation is a disease of the central nervous system.

4. A method in accordance with claim 3, wherein the disease is Alzheimer's disease.

5. In a method for the treatment of a disease characterized by amyloid aggregation in a patient by causing antibodies against a peptide component of an amyloid deposit to come into contact with aggregated or soluble amyloid, the improvement by which risk of inflammation is diminished, comprising:
eliminating the inflammatory pathway initiated by binding of an immune complex to an Fc receptor by eliminating the Fc regions of said antibodies or causing the Fc receptors to be blocked.

6. A method in accordance with claim 5, wherein said step of eliminating the inflammatory pathway comprises using as said antibodies, antibodies devoid of Fc regions.

7. A method in accordance with claim 6, wherein said antibodies are Fab, F(ab)2 and/or scFv antibodies.

8. A method in accordance with claim 6, wherein said antibodies are presented on a carrier which potentiates efflux of the antibody amyloid complex.

9. A method in accordance with claim 8, wherein said carrier is a filamentous phage.

10. A process in accordance with claim 6, wherein the disease characterized by amyloid aggregation is a disease of the central nervous system.

11. A process in accordance with claim 10, wherein the disease is Alzheimer's disease.

12. A method in accordance with claim 5, wherein said step of eliminating the inflammatory pathway comprises causing the Fc receptors to be blocked.

13. A method in accordance with claim 12, wherein said causing of the Fc receptors to be blocked is accomplished by intravenously administering an effective amount of immunoglobulin (IVIg) to the patient prior to causing the antibodies to come into contact with the amyloid.

14. A method in accordance with claim 13, wherein the IVIg is intact human polyclonal immunoglobulin.

14. A method in accordance with claim 12, wherein the disease characterized by amyloid aggregation is a disease of the central nervous system.

16. A method in accordance with claim 15, wherein the disease is Alzheimer's disease.
